(19) United States
(12) Patent Application Publication
(10) Pub. No.: US 2019/0167595 A1
(43) Pub. Date: Jun. 6, 2019

(54) COMPOSITIONS OF PHARMACEUTICAL PRODUCT WITH INGESTIBLE EVENT MARKER

(71) Applicant: Otsuka Pharmaceutical Co., Ltd., Tokyo (JP)

(72) Inventors: Mark ZDEBLICK, Portola Valley, CA (US); Markus CHRISTEN, Hillsborough, CA (US); Nikhil PARGAONKAR, Hayward, CA (US); Nikolaus LEIST, San Carlos, CA (US); Kurt SCHEINPFLUG, Dublin, CA (US); Patricia JOHNSON, San Carlos, CA (US); Benedict COSTELLO, Piedmont, CA (US)

(73) Assignee: Otsuka Pharmaceutical Co., Ltd., Tokyo (JP)

(21) Appl. No.: 16/170,418

(22) Filed: Oct. 25, 2018

Related U.S. Application Data
(63) Continuation of application No. PCT/JP2017/016395, filed on Apr. 25, 2017.
(60) Provisional application No. 62/327,418, filed on Apr. 25, 2016.

(51) Int. Cl. A61K 9/20; A61K 31/496; A61B 5/07

(52) U.S. Cl. CPC A61K 9/2009; A61B 5/07; A61K 31/496

(57) ABSTRACT

In some embodiments, a composition of matter includes aripiprazole, a metal selected from the group consisting of magnesium, zinc, sodium, lithium, iron, or alloys thereof, or combinations thereof and a copper salt selected from the group consisting of copper iodide, copper chloride, copper bromide, copper sulfate, copper formate, or combinations thereof.

### CROSS-REFERENCE TO RELATED APPLICATIONS

This application claims priority to and is a continuation of PCT Application No. PCT/JP2017/016395, filed Apr. 25, 2017, and titled “Compositions of Pharmaceutical Product with Ingestible Event Marker,” which claims priority to and the benefit of U.S. Provisional Patent Application No. 62/327,418, filed Apr. 25, 2016, and titled “Compositions of Pharmaceutical Product with Ingestible Event Marker,” which is incorporated herein by reference in its entirety.

### INTRODUCTION

Prescription medications are effective remedies for many patients when taken as instructed by the prescribing physician. However, studies have shown that, on average, about 50% of patients do not comply with prescribed medication regimens. A low rate of compliance with medication regimens results in a large number of hospitalizations and admissions to nursing homes every year. In the United States alone, it has recently been estimated that the cost resulting from patient non-compliance is reaching $100 billion annually.

One example situation where patient adherence is of particular importance is in the context of clinical studies. Non-adherence in the clinical trial setting has long-range consequences far beyond the few hundred patients who might be involved in a trial. To the extent that non-adherence occurs without a correction factor, it may have effects ranging from failure to gain Food and Drug Administration (FDA) approval to the necessity for increasing the recommended dose beyond that which would be required of a fully compliant population. Such an elevated dose could cause a higher incidence of side effects, which in turn may lead to further non-adherence.

Clinical studies typically enroll patients to undergo specific drug treatment regimens with the goal of testing hypotheses related to the effects of drug treatment on medically relevant clinical endpoints. Such studies might measure, for example, the relationship between alternative drug treatments with any of a wide variety of clinical endpoints, ranging from physiological, biochemical or psychological measurements, to manifestations of disease, patient survival or quality of life. In addition, drug treatments must also be related to any observed adverse events in an effort to identify rare adverse reactions or interactions with other medications.

The ability to reliably correlate highly specific drug treatment regimens, including dosage and administration methods, with both efficacy and safety depends to a great extent on the certainty of knowledge that every patient has followed the prescribed treatment regimen. Monitoring of patient adherence, including the exact time of administration for medications, is therefore of great value to patients and their physicians, as well as clinical trial sponsors and the pharmaceutical industry in general.

Various methods and apparatus have been made available to improve patient compliance with prescribed regimens in efforts to improve patient health. Transdermal delivery systems combined with a unique biologically active ingredients such as aripiprazole, risperidone, quetiapine, and brexpiprazole provide sustained release formulations for the safe and efficacious transdermal administration of the unique biologically active ingredient to a subject through a body surface or membrane over a sustained time period for the treatment of various diseases. The transdermal route of parenteral delivery of drugs and other biologically active ingredients (“agents”) has been proposed for a wide variety of systemically acting and locally acting agents on either a rate-controlled or non-rate-controlled basis. For example, sustained release formulations for the safe and efficacious administration of pharmaceutical active ingredients for the treatment of hypertension, congestive heart failure, and acute and chronic renal failure, among other things, have been proposed.

Additionally, different types of “smart” packaging devices have been developed. In some cases, such devices automatically dispense the appropriate pill. In other cases, there are electronic controls that detect and record when the pill is taken out of the box. However, improvements of patient compliance with prescription regimens have not addressed automatic tracking of oral administration (e.g., ingestion) of active ingredient to a patient in need of administration thereof.

Thus, provided herein are methods of oral administration of an active ingredient (e.g., aripiprazole, risperidone, quetiapine, or brexpiprazole) with an electronic circuitry system such as the electronic circuitry system developed by Proteus Digital Health, Inc. described in U.S. Pat. Nos. 7,978,064; 8,674,825; 8,730,031; 8,802,183; 8,816,847; 8,836,513; 8,847,766; and 8,912,908, the disclosures of which are incorporated in their entirety herein by reference. Also provided herein are delivery systems employing electronic circuits combined with specific formulations of active ingredients (e.g., aripiprazole, risperidone, quetiapine, and brexpiprazole) to provide different techniques for tracking oral delivery of the active ingredient to a patient in need of administration of an active ingredient such as aripiprazole, risperidone, quetiapine, or brexpiprazole.

The present disclosure provides a unique composition of matter comprising the combination of the electronic circuitry comprising battery forming materials and specific formulations of an active ingredient as described herein to confirm the delivery of the specific formulations of active ingredient. The present novel composition of matter also overcomes the unpredictable nature of combining various metals and salts with specific formulations of aripiprazole, risperidone, quetiapine, or brexpiprazole to provide an electronic delivery system that generates its own electrical power from a partial energy source comprised of dissimilar materials when exposed with the bodily fluids of a patient during the oral administration of specific formulations of aripiprazole, risperidone, quetiapine, or brexpiprazole.

The present disclosure relates generally to a composition of matter for the active monitoring of the ingestible administration of an active ingredient. The composition of matter includes the active ingredient (e.g., aripiprazole, risperidone, quetiapine, or brexpiprazole) magnesium metal and copper chloride. These materials and the final complete tablet formulation were chosen for a variety of reasons. First, we were able to show that this specific formulation of copper chloride, magnesium metal, connected by silicon that is conductive when wet, do not appreciably alter the chemical composition and ultimately the bioavailability of the active ingredient when ingested even after being stored after manufacturing for an extended period of time. Second, the combination of the active ingredient with copper chloride, magnesium metal and silicon does not facilitate the reaction of copper chloride and magnesium metal. Such a reaction, for example while being stored after manufacturing and before delivery to a patient, could cause the magnesium metal or copper chloride to react and become larger in size, exerting significant forces on the surrounding active ingredient as formulated and causing the tablet to break up into pieces; the bi-products of such a reaction could change the chemical composition of the active ingredient; or, if all or most of the magnesium metal or copper chloride are reacted, render the ingestion sensor powerless and inert when ingested. Thus, uniquely, a formulation containing the active ingredient and the materials that make up the ingestion sensor must be found and proven to not adversely affect the purpose of the other.

An example of how this conflict has manifested itself in early experiments demonstrates this unique challenge: Early experiments were made without any active ingredient in the tablet: just the ingestion sensor and “placebo” formulation of inert materials. Now, a placebo pill without an embedded ingestion sensor can sit in an open container in a hot, humid bathroom for months without changing its ultimate performance. Many—but not all—pharmaceuticals or dietary supplements such as vitamins can be stored in a similar manner without adversely affecting their effectiveness. When in our early experiments we added ingestion sensors to such “placebo” vitamin tablets, however, we found that the partial power source made of copper chloride (or other salts) and magnesium metal (or other metals) would react with each other—effectively “discharging” the biogalvanic potential—before the vitamin-with-ingestion-sensor tablet was ingested. Further, with some active ingredients, the change in size of either the copper chloride or magnesium metal, when reacted, would cause some of the tablet to break up into pieces. Thus, the process of discovering and validating a precise formulation including active ingredient, magnesium metal, copper chloride and silicon that allows all of these materials to co-exist for an extended period of time in an uncertain storage environment is a unique challenge that depends upon the reactivity of the active ingredient as formulated with the pair of electrochemically active materials, magnesium metal and copper chloride. More specifically, the present disclosure relates to compositions used in an apparatus for automatic identification of ingestion or oral administration of active ingredient.

### SUMMARY

According to one aspect of the present disclosure, a composition of matter for the ingestible administration of active ingredient (e.g., aripiprazole, risperidone, quetiapine, or brexpiprazole) is provided. In some embodiments, the composition comprises an active ingredient (such as aripiprazole, risperidone, quetiapine, or brexpiprazole); a metal including at least one of magnesium metal, zinc, sodium, lithium, iron or alloys thereof, or combinations thereof, a copper salt including at least one of copper iodide, copper chloride, copper bromide, copper sulfate, copper formate, or combinations thereof, and silicon.

According to one aspect of the present disclosure, an apparatus is provided. The apparatus comprises active ingredient (as described herein); a substrate with a first surface and a second surface; a partial power source comprising a first material provided on the first surface of the substrate, wherein the first material is selected from the group consisting of magnesium metal, zinc, sodium, lithium, iron, and alloys thereof, an intercalation compound, vanadium oxide, manganese oxide, and combinations thereof and a second material provided on the second surface of the substrate, wherein the second material is selected from copper iodide, copper chloride, copper bromide, copper sulfate, copper formate, iron(III) phosphate, iron(III) pyrophosphate, oxygen, hydrogen, vanadium oxide, manganese oxide, and combinations thereof, wherein the partial power source is configured to generate power upon contact of the first material and the second material with a fluid; and a control unit electronically coupled with the partial power source, wherein the control unit is configured to be activated by receiving the power from the partial power source and to encode information in a current flow through the fluid.

According to another aspect of the present disclosure, a composition of matter for the ingestible administration of active ingredient is provided. The composition comprises one of aripiprazole, risperidone, quetiapine, or brexpiprazole; and silicon having a mass equivalent to a silicon substrate having dimensions of between 0.5×0.5×0.5 mm (0.125 mm³) and 3×3×1 mm (09 mm³), or more particularly, roughly 1.0×1.0×0.3 mm (0.3 mm³).

### BRIEF DESCRIPTION OF THE FIGURES

The features of the various aspects of the present disclosure are set forth with particularity in the appended claims. The various aspects, both as to organization and methods of operation, together with advantages thereof, may, however best be understood by reference to the following description, taken in conjunction with the accompanying drawings as follows:

FIG. 1 is a diagrammatic, exemplary representation of the pill embodiment of the present disclosure, according to one aspect of the present disclosure.

FIG. 2 is a more detailed view of the pill composition shown in FIG. 1, according to one aspect of the present disclosure.

FIG. 3 is an example embodiment of signal generation elements of the pill composition shown in FIG. 1, according to one aspect of the present disclosure.

FIG. 4 is an example embodiment of signal generation elements of the pill composition shown in FIG. 1, according to one aspect of the present disclosure.

FIG. 5 is an assembling apparatus for assembling a signal generation element on a tablet, according to one aspect of the present disclosure.

FIG. 6 is a close-up view of a portion of the apparatus of FIG. 5 with specific indication of the direction of force applied, according to one aspect of the present disclosure.

FIG. 7 is a close-up view of a portion of a feeder assembly of the apparatus of FIG. 5, according to one aspect of the present disclosure.

FIG. 8 is a close-up view of a portion of a feeder assembly that can be used with the apparatus of FIG. 5 in accordance with another aspect of the present disclosure.

FIG. 9A is a close-up view of a portion of a feeder assembly that can be used with the apparatus of FIG. 5 in accordance with another aspect of the present disclosure.

FIG. 9B is a close-up view of a portion of the feeder assembly shown in FIG. 9A at an advanced stage in the loading process, according to one aspect of the present disclosure.

FIG. 10 shows lot release testing Activation Time data for target hardness active ingredient IEM (Ingestible Event Marker) tablet lots, according to one aspect of the present disclosure.

FIG. 11 shows lot release testing Life Time data for target hardness active ingredient IEM tablet lots, according to one aspect of the present disclosure.

FIG. 12 shows lot release testing Mean Amplitude data for target hardness active ingredient IEM tablet lots, according to one aspect of the present disclosure.

FIG. 13 shows a concave beveled edge tablet press tool set, according to one aspect of the present disclosure.

FIG. 14 shows an upper tablet press punch, according to one aspect of the present disclosure.

FIG. 14A is a detail view of the upper tablet press punch shown in FIG. 14, according to one aspect of the present disclosure.

FIG. 15 shows a lower tablet press punch, according to one aspect of the present disclosure.

FIG. 16 shows a plan view of a tablet press die, according to one aspect of the present disclosure.

FIG. 17 is partial section view taken along section line 17 of the tablet press die shown in FIG. 16, according to one aspect of the present disclosure.

### DETAILED DESCRIPTION

The drawings and descriptions provided herein should be regarded as illustrative in nature and not restrictive.

Any one or more of the teachings, expressions, aspects, examples, etc. described herein may be combined with any one or more of the other teachings, expressions, aspects, examples, etc. that are described herein. The following described teachings, expressions, aspects, examples, etc. should, therefore, not be viewed in isolation relative to each other. Various suitable ways in which the teachings herein may be combined will be readily apparent to those of ordinary skill in the art in view of the teachings herein. Such modifications and variations are intended to be included within the scope of the claims.

The present disclosure provides the clinician an important new tool in their therapeutic armamentarium: automatic detection and identification of pharmaceutical agents actually delivered into the body. The applications of this new information device and system are multi-fold. By example, when used in concert with other medical sensing devices, correlation between drug delivery, batch and dosage can be correlated to a physiological response. In this manner, optimal pharma-therapeutic regimens may be formulated by the clinician.

Assessment of medications is made possible by the present disclosure without resort to awaiting overt clinical sequel of treatment, many of which can be seriously adverse. By example, positive effects would be quickly ascertainable without being obscured by more random factors. Negative responses, such as changes in blood pressure, would become clearly evident as drug related or independent above background physiologic variation.

The ability to document the ingestion of a drug or other actual exposure of the body to a medication has many important clinical applications. In the simplest form, this technique provides accurate data of when a pill has been taken and which pill has been taken. This allows the precise determination of which pill was taken at a specific point in time. Such monitoring capability assures patients are taking the prescribed medication correctly. This information avoids the potential for over prescription of medications that are not actually being taken.

The present disclosure provides the clinician an accurate dose response curve showing the response to a medication and the timing of the ingestion of the pill. Such data has many applications. For instance, the clinician now has the ability to determine which patients have no response to the medicine in the pill. In a study situation, such patients can be removed from a study or a test of the clinical utility of a certain medication. This provides that only people who have a beneficial response to a certain medication are retained in the trial. This feature will improve the efficacy of medications and to reduce the amount of medications that people take that are not being useful. It may also be used in trials to determine which patients actually consumed the medicine, and which did not.

In more standard clinical environments, this unique data allows careful selection and titration of drug administration without resorting to more overt physical symptoms to ascertain contraindications, efficacy, and optimal dosage levels. The present disclosure provides a record for emergency room technicians or doctors when a patient is admitted to a hospital so that the patient's status can be accurately ascertained. Dosage events within the last hour or day prior to admission, and the identity of the last medication, will be immediately available.

The clinician obtains this information through simple interrogation of the implanted or portable device. This device would tell them without any uncertainty what pills have been taken.

A “smart box” may be provided that can interrogate each pill and ascertain its address. The box can write a distinctive product number or product code so that every single pill ever made is provided with a unique identifier. Fuses, for example, may be selectively destroyed so the addresses may be detected electrically or optically. The present disclosure makes it possible to identify precisely who bought such a pill from the authorized pharmacist.

Embodiments of the disclosure may include compositions having an identifier stably associated therewith. In certain embodiments, the compositions may be disrupted upon administration to a subject. As such, in certain embodiments, the compositions may be physically broken, e.g., dissolved, degraded, eroded, etc., following delivery to a body, e.g., via ingestion. The compositions of these embodiments may be distinguished from devices that are configured to be ingested and survive transit through the gastrointestinal tract substantially, if not completely, intact. While the compositions of these embodiments may be themselves disrupted upon administration, components of the composition, e.g., the identifier, may survive transit of the gastrointestinal tract, e.g., as described in greater detail below.

In certain embodiments, the compositions may include an active ingredient/carrier component and an identifier. Each of these different components is reviewed separately in greater detail below.

### Active Ingredient/Carrier Component

The subject compositions may include an active ingredient/carrier component. The active ingredient/carrier component may be a solid, which has an amount of active ingredient, e.g., a dosage, present in a pharmaceutically acceptable carrier. The active ingredient/carrier component may be referred to as a “dosage formulation.”

### Active Ingredient

An “active ingredient” produces a physiological result, e.g., a beneficial or useful result, upon contact with a living organism, e.g., a mammal, such as a human. Compositions provided herein comprise aripiprazole, risperidone, quetiapine, or brexpiprazole, and may be referred to as aripiprazole, risperidone, quetiapine, or brexpiprazole, their respective IUPAC names (e.g., 7-(4-(4-(2,3-dichlorophenyl)-1-piperazinyl)butyloxy)-3,4-dihydro-2(1H)-quinolinone, 3-[2-[4-(6-fluoro-1,2-benzoxazol-3-yl)piperidin-1-yl]ethyl]-2-methyl-6,7,8,9-tetrahydropyrido[1,2-a]pyrimidin-4-one, 2-[2-(4-benzo[b][1,4]benzothiazepin-6-ylpiperazin-1-yl)ethoxy]ethanol, 7-[4-[4-(1-benzothiophen-4-yl)piperazin-1-yl]butoxy]-1H-quinolin-2-one) or brand names (Abilify®, Risperdal®, Seroquel®, Rexulti®).

In some embodiments, the active ingredient as used herein may be present as a pharmaceutically acceptable salt (e.g., a pharmaceutically acceptable salt found in Remington's Pharmaceutical Sciences, Mace Publishing Company, Philadelphia, Pa., 17th ed. 1985).

As indicated above, in some embodiments the active ingredient of the compositions provided herein may be present in a pharmaceutically acceptable vehicle or carrier, e.g., as described below. In some embodiments, the active ingredient may be present in an amount of from about 0.1% to about 90% by weight, e.g., from about 1% to about 30% by weight of the compositions.

### Pharmaceutically Acceptable Carrier

In some embodiments, the compositions provided herein comprise excipients as described herein added to the active ingredient and compacted at 7000-16500 psi of pressure.

As summarized above, the compositions of the disclosure may further include a pharmaceutically acceptable vehicle (i.e., carrier). Common carriers and excipients, such as corn starch or gelatin, lactose, dextrose, sucrose, microcrystalline cellulose, kaolin, mannitol, dicalcium phosphate, sodium chloride, and alginic acid may be of interest. Disintegrators commonly used in the formulations of the disclosure may include croscarmellose, microcrystalline cellulose, corn starch, sodium starch glycolate and alginic acid.

A liquid composition may comprise a suspension or solution of the compound or pharmaceutically acceptable salt in a suitable liquid carrier(s), for example, ethanol, glycerin, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder. For example, a powder containing active compound, suspending agent, sucrose and a sweetener can be reconstituted with water to form a suspension; and a syrup can be prepared from a powder containing active ingredient, sucrose and a sweetener.

A composition in the form of a tablet or pill can be prepared using any suitable pharmaceutical carrier(s) routinely used for preparing solid compositions. Examples of such carriers include magnesium stearate, starch, lactose, sucrose, microcrystalline cellulose and binders, for example, polyvinylpyrrolidone. The tablet can also be provided with a color film coating, or color included as part of the carrier(s). In addition, active compound can be formulated in a controlled release dosage form as a tablet comprising a hydrophilic or hydrophobic matrix. After extensive experimentation and validation studies, we found that hydroxypropyl cellulose acted as the binder that, combined with the active ingredient, produced an ingestion sensor that retained functionality after one year of storage.

“Controlled release”, “sustained release”, and similar terms are used to denote a mode of active ingredient delivery that occurs when active ingredient (as described herein) is released from the delivery vehicle at an ascertainable and controllable rate over a period of time, rather than dispersed immediately upon ingestion. Controlled or sustained release may extend for a period of time (e.g., minutes or hours), and may vary as a function of numerous factors. For the pharmaceutical composition of the present disclosure, the rate of release will depend on the type of the excipient selected and the concentration of the excipient in the composition. Another determinant of the rate of release is the rate of hydrolysis of the linkages between and within the units of a polyorthoester, which, in some embodiments is included in the compositions provided herein. The rate of hydrolysis in turn may be controlled by the composition of the polyorthoester and the number of hydrolysable bonds in the polyorthoester. Other factors determining the rate of release of active ingredient from the present pharmaceutical composition include particle size, acidity of the medium (either internal or external to the matrix) and physical and chemical properties of the active ingredient in the matrix. After extensive experimentation and validation studies, we found that magnesium stearate acted as the release agent that, combined with the active ingredient, produced a functional ingestion sensor after one year of storage.

After extensive experimentation and validation studies, we found that hydroxypropyl cellulose acted as the binder that, combined with an active ingredient as described herein, produced a functional ingestion sensor after one year of storage.

It may be desirable to add a coloring agent to make the dosage form more attractive in appearance or to help identify the product. We have discovered, however, that some colors comprise copper compounds, and thus affect the copper load of the overall composition. Thus, the color of tablet must be chosen with care to prevent undesirable effects on the overall clinical and regulatory performance of the ingestion sensor comprising an active ingredient as described herein. After extensive experimentation and validation studies, we found that ferric oxide (yellow or red) and FD&C Blue No. 2 Aluminum Lake acted as the coloring agent that, combined with an active ingredient as described herein, produced a functional ingestion sensor after one year of storage.

### Identifiers

Also present in the subject compositions is an identifier. The identifier may vary depending on the particular embodiment and intended application of the composition. In certain embodiments, the identifier may be a component that emits a signal upon activation by a stimulus, e.g., by interrogation, upon contact with a target physiological location, etc. As such, the identifier may be an identifier that emits a signal when it contacts a target body (i.e., physiological) site. In addition or alternatively, the identifier may be an identifier that emits a signal when interrogated.

In yet other embodiments, the identifier may be an inert, but identifiable marker, e.g., an engraved identifier (such as one that is fabricated from a material or materials that survive digestion). This marker may then be identified, for example, following an autopsy or forensic examination. It is possible to provide a more internal device within a pill to determine both that its surface has partially been subject to digestion, but also that the inner pill material has also been digested. This application may be particularly useful in experimental pharmacological settings. The identifier of these embodiments may be one that does not necessarily emit a signal, but which can be optically inspected, e.g., visually or machine read, to obtain information about the composition with which it was associated prior to administration.

While the identifier may be an identifier that does not emit a signal, in certain embodiments, as summarized above, the identifier may be one that does emit a signal. Depending on the needs of a particular application, the signal may be a generic signal, e.g., a signal that merely identifies that the composition has contacted the target site, or a unique signal, e.g., a signal which in some way uniquely identifies that a particular composition from a group or plurality of different compositions in a batch has contacted a target physiological site. As such, the identifier may be one that, when employed in a batch of unit dosages, e.g., a batch of tablets, may emit a signal which cannot be distinguished from the signal emitted by the identifier of any other unit dosage member of the batch. In yet other embodiments, the identifier may emit a signal that uniquely identifies a given unit dosage, even from other identical unit dosages in a given batch. Accordingly, in certain embodiments, the identifier may emit a unique signal that distinguishes a given type of unit dosage from other types of unit dosages, e.g., a given medication from other types of medications. In certain embodiments, the identifier may emit a unique signal that distinguishes a given unit dosage from other unit dosages of a defined population of unit dosages, e.g., a prescription, a batch or a lifetime production run of dosage formulations. In certain embodiments, the identifier may emit a signal that is unique, i.e., distinguishable, from a signal emitted by any other dosage formulation ever produced, where such a signal may be viewed as a universally unique signal (e.g., analogous to a human fingerprint which is distinct from any other fingerprint of any other individual and therefore uniquely identifies an individual on a universal level). In one embodiment, the signal may either directly convey information about the composition, or provide an identifying code, which may be used to retrieve information about the composition from a database, i.e., a database linking identifying codes with compositions.

The identifier may be any component or device that is capable of generating a detectable signal following activation in response to a stimulus. In certain embodiments, the stimulus may activate the identifier to emit a signal once the composition comes into contact with a physiological target site, e.g., as summarized above. For example, a patient may ingest a pill that upon contact with the stomach fluids, generates a detectable signal. Depending on the embodiment, the target physiological site or location may vary, where representative target physiological sites of interest include, but are not limited to: a location in the gastrointestinal tract (such as the mouth, esophagus, stomach, small intestine, large intestine, etc.); another location inside the body, such as a parental location, vascular location, etc.; or a topical location; etc.

In certain embodiments, the stimulus that activates the identifier may be an interrogation signal, such as a scan or other type of interrogation. In these embodiments, the stimulus may activate the identifier, thereby emitting a signal which may then be received and processed, e.g., to identify the composition in some manner.

In certain of these embodiments, the identifier may include a power source that transduces broadcast power and a signal generating element that modulates the amount of transduced power, such that a signal is not emitted from the identifier but instead the amount of broadcast power transduced by the identifier is detected and employed as the “signal.” Such embodiments may be useful in a variety of applications, such as applications where the history of a given composition may be of interest, e.g., as reviewed in greater detail below.

In certain embodiments, the identifier may be dimensioned to be complexed with the active ingredient/pharmaceutically acceptable carrier component to produce a composition that can be readily administered to a subject in need thereof. As such, in certain embodiments, the identifier element may be dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm. In certain embodiments, the identifier may be 1 mm3 or smaller, such as 0.1 mm3 or smaller, including 0.2 mm3 or smaller. The identifier element may take a variety of different configurations, such as but not limited to: a chip configuration, a cylinder configuration, a spherical configuration, a disc configuration, etc., where a particular configuration may be selected based on intended application, method of manufacture, etc.

The identifier may generate a variety of different types of signals, including but not limited to, RF, magnetic, conductive (near field), acoustic, etc.

In certain embodiments, the identifier may be one that is programmable following manufacture, in the sense that the signal generated by the identifier may be determined after the identifier is produced, where the identifier may be field programmable, mass programmable, fuse programmable, and even reprogrammable. Such embodiments are of interest where uncoded identifiers are first produced and following incorporation into a composition are then coded to emit an identifying signal for that composition. Any convenient programming technology may be employed. In certain embodiments, the programming technology employed is RFID technology. RFID smart tag technology of interest that may be employed in the subject identifiers includes, but is not limited to: that described in U.S. Pat. Nos. 7,035,877; 7,035,818; 7,032,822; 7,031,946, as well as published application no. 20050131281, and the like, the disclosures of which are herein incorporated by reference. With RFID or other smart tag technology, a manufacturer/vendor may associate a unique ID code with a given identifier, even after the identifier has been incorporated into the composition. In certain embodiments, each individual or entity involved in the handling of the composition prior to use may introduce information into the identifier, e.g., in the form of programming with respect to the signal emitted by the identifier, e.g., as described in U.S. Pat. No. 7,031,946 the disclosure of which is herein incorporated by reference.

The identifier of certain embodiments may include a memory element, where the memory element may vary with respect to its capacity. In certain embodiments, the memory element may have a capacity ranging from about 1 bit to 1 gigabyte or more, such as 1 bit to 1 megabyte, including from about 1 bit to about 128 bit. The particular capacity employed may vary depending on the application, e.g., whether the signal is a generic signal or coded signal, and where the signal may or may not be annotated with some additional information, e.g., name of the active ingredient, etc.

Identifier components of embodiments of the disclosure may have: (a) an activation component and (b) a signal generation component, where the signal generation component is activated by the activation component to produce an identifying signal, e.g., as described above.

### Activation Component

The activation component may be a component that activates the signal generation element to emit a signal upon experience of a stimulus, e.g., contact of the composition with a target physiological site of interest, such as the stomach. The activation component may be integrated with a power source, e.g., a battery. Illustrative activation approaches may include, but are not limited to: Battery Completion, e.g., Battery activated by electrolyte addition and Battery activated by cathode or anode addition; Battery connection, e.g., Battery activated by conductor addition; Transistor-mediated Battery Connection, e.g., Battery activated by transistor gate, Geometry Modification, Detection of Geometry Modification by Resonant Structure, Pressure Detection, Resonant Structure Modification; etc.

### Battery/Power Source

In certain embodiments, the power source may be turned on upon contact of the power source with a target site, e.g., a physiological target site, such as the stomach, e.g., stomach acid. In certain embodiments, the power source may be a battery that is turned on to provide power upon contact with the physiological target site, where the battery is coupled to the signal generation component such that when the battery is turned on, the signal generation component may emit the identifying signal.

In certain embodiments, the battery that is employed may be one that comprises the two dissimilar materials magnesium metal and copper chloride, which constitute the two electrodes of the battery. In certain embodiments, these two materials may be shielded from the surrounding environment by an additional layer of material. When the shielding material (e.g., active ingredient/carrier matrix), is dissolved or eroded by the surrounding fluid, the electrode materials may be exposed and come in contact with the body fluid, such as stomach acid or other types of electrolyte fluid. A potential difference, that is, a voltage, may be generated between the electrodes as a result of the respective oxidation and reduction reactions incurred to the two electrode materials. A voltaic cell, or battery, can be thereby formed. Accordingly, in some embodiments of the disclosure, such batteries may be configured such that when the two dissimilar materials are exposed to the target site, e.g., the stomach, the digestive tract, etc., during the physical and chemical erosion of the composition in which the signal generation element is present, a voltage may be generated. In such embodiments, the power source described above is not a “battery” in the common sense of the word, but rather as defined in the discipline of physics. The two dissimilar materials (magnesium metal and copper chloride) in an electrolyte may be at different potentials. As a result, a potential difference between the two dissimilar materials may be generated.

Various battery-activation configurations are possible. Representative types of cell-activation approaches may include, but are not limited to: activation by presence of electrolyte, activation by presence of a cathode material, activation by presence of a conductive material.

After the battery is activated, further activation configurations can be employed to activate the signal generation component. For example, the signal generation component can be activated through the activation of the gate of a metal oxide semiconductor (MOS) circuit, such as a CMOS switch. Activation of the gate of the MOS circuit can be based on one or more parameters, which may include but are not limited to: gate current, gate charge, and gate capacitance.

The gate current, for activation purposes, can be a function of the conductivity of surrounding body fluids or tissues. Such conductivity can further be a function of one or more parameters, which may include but are not limited to: solution concentration, solution pH value, ionic content of solution, enzymatic content of solution, temperature, and carrier mobility. Carrier mobility can also be a function of temperature.

Similarly, the gate charge can be a function of one or more parameters, which may include but are not limited to: solution composition, crystal potential, electrical potential, gravitational potential, gate capacitance, and carrier concentration. The carrier concentration can also be a function of temperature.

The gate capacitance can be a function of the capacitive geometry of the gate, which can further be a function of pressure, a resonant input, or the characteristics of a dielectric material coupled to the gate. The characteristics of the dielectric material can vary with one or more parameters, which may include but are not limited to: chemical contents of a digestive tract, chemical character of a physiological location, and amount of dissolution of the dielectric material in body fluids.

In certain embodiments, the battery may be one that is made up of active electrode, electrolyte, and inactive materials, such as current collectors, packaging, etc. The active materials are a pair made up of magnesium metal and copper chloride.

The electrode materials provided herein are copper chloride as the anode and magnesium metal as the cathode.

Some embodiments of the batteries described herein provide for a voltage upon contact with the target physiological site, e.g., the stomach, sufficient to drive the signal generation element of the identifier. In certain embodiments, the voltage provided by the electrode materials upon contact of the metals of the power source with the target physiological site may be 0.001 V or higher, including 0.01 V or higher, such as 0.1 V or higher, e.g., 0.3 V or higher, including 0.5 volts or higher, and including 1.0 volts or higher, where in certain embodiments, the voltage may range from about 0.001 to about 10 volts, such as from about 0.01 to about 10 V.

In certain embodiments, the batteries may have a small form factor. Batteries may be 10 mm³ or smaller, such as 1.0 mm³ or smaller, including 0.1 mm³ or smaller, including 0.02 mm³ or smaller. As such, in certain embodiments, the battery element is dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm.

In certain embodiments, the battery may have a split or segmented configuration.

In certain embodiments, the battery may be one free of packaging. As such, the electrodes may be exposed and not protected by any protecting or sealing structure. As such, following removal of the active ingredient/carrier matrix material with which the battery may be associated, the battery per se does not itself include a protective packaging such that the electrodes may be free to contact the electrolyte at the target physiological location.

In certain embodiments, the battery power source may be viewed as a power source that exploits reverse electrolysis in an ionic solution such as gastric fluid, blood, or other bodily fluids and some tissues.

Where the power source is a battery, the battery may be fabricated in a number of different ways. In certain embodiments, fabrication protocols which may be categorized as “planar” processing protocols are employed, as developed in greater detail below.

### Signal Generation Component

The signal generation component of the identifier element is a structure that, upon activation by the activation component, may emit a detectable signal, e.g., that can be received by a receiver. The signal generation component of certain embodiments can be any convenient device that is capable of producing a detectable signal and/or modulating transduced broadcast power, upon activation by the activation component. Detectable signals of interest include, but are not limited to: conductive signals, acoustic signals, etc. As reviewed above, the signals emitted by the signal generator may be generic or unique signals, where representative types of signals of interest include, but are not limited to: frequency shift coded signals; amplitude modulation signals; frequency modulation signals; etc.

In certain embodiments, the signal generation element may include circuitry which produces or generates the signal. The type of circuitry chosen may depend, at least in part, on the driving power that is supplied by the power source of the identifier. For example, where the driving power is 1.2 volts or above, standard CMOS circuitry may be employed. In other embodiments where the driving power ranges from about 0.7 to about 1.2 V, sub-threshold circuit designs may be employed. For driving powers of about 0.7 V or less, zero-threshold transistor designs may be employed.

In certain embodiments, the signal generation component includes a voltage-controlled oscillator (VCO) that can generate a digital clock signal in response to activation by the activation component. The VCO can be controlled by a digital circuit, which is assigned an address and which can control the VCO with a control voltage. This digital control circuit can be embedded onto a chip that includes the activation component and oscillator. Using phase shift keying to encode the address, an identifying signal can be transmitted.

The signal generation component may include a distinct transmitter component that serves to transmit the generated signal to a remote receiver, which may be internal or external to the patient, as reviewed in greater detail below. The transmitter component, when present, may take a number of different configurations, e.g., depending on the type of signal that is generated and is to be emitted. In certain embodiments, the transmitter component may be made up of one or more electrodes. In certain embodiments, the transmitter component may be made up of one or more wires, e.g., in the form of antenna(e). In certain embodiments, the transmitter component may be made up of one or more coils. As such, the signal transmitter may include a variety of different transmitters, e.g., electrodes, antennas (e.g., in the form of wires) coils, etc. In certain embodiments, the signal may be transmitted either by one or two electrodes or by one or two wires. A two-electrode transmitter may be a dipole; a one electrode transmitter forms a monopole. In certain embodiments, the transmitter may only require one diode drop of power.

### Additional Components

Depending on the particular embodiment, the identifier may include a number of different additional components. Some components of interest include, but are not limited, those reviewed below.

### Power Enhancers

Where the activator is a power source that is turned on upon contact with a target physiological site, in certain embodiments, circuits for enhancing or boosting voltage of the analog circuit voltage rails, may be provided, e.g., charge pumping circuits, charge doublers, etc. By increasing the voltage of certain nodes, improved performance of critical functions, such as oscillators, can be achieved.

### Power Storage

In certain embodiments, the activation component may include a power storage element. For example, a duty cycle configuration may be employed, e.g., where slow energy production from a battery is stored in a power storage element, e.g., in a capacitor, which then may provide a burst of power that may be deployed to the signal generation component. In certain embodiments, the activation component may include a timing element which modulates, e.g., delays, delivery of power to the signal generation element, e.g., so signals from different compositions, e.g., pills, that are administered at substantially the same time may be produced at different times and are therefore distinguishable.

### Identifier Fabrication

In certain embodiments of interest, the identifier element includes a semiconductor support component. Any of a variety of different protocols may be employed in manufacturing the identifier structures and components thereof. For example, molding, deposition and material removal, e.g., planar processing techniques, such as Micro-Electro-Mechanical Systems (MEMS) fabrication techniques, including surface micromachining and bulk micromachining techniques, may be employed. Deposition techniques that may be employed in certain embodiments of fabricating the structures include, but are not limited to: electroplating, cathodic arc deposition, plasma spray, sputtering, e-beam evaporation, physical vapor deposition, chemical vapor deposition, plasma enhanced chemical vapor deposition, etc. Material removal techniques include, but are not limited to: reactive ion etching, anisotropic chemical etching, isotropic chemical etching, planarization, e.g., via chemical mechanical polishing, laser ablation, electronic discharge machining (EDM), etc. Also of interest are lithographic protocols. Of interest in certain embodiments is the use of planar processing protocols, in which structures are built up and/or removed from a surface or surfaces of an initially planar substrate using a variety of different material removal and deposition protocols applied to the substrate in a sequential manner.

### Specific Pill Embodiments

In further describing various embodiments of the compositions of the disclosure, specific embodiments are now described in greater detail in view of the figures. In the following detailed description, reference is made to the accompanying drawings, which form a part hereof. In the drawings, similar symbols and reference characters typically identify similar components throughout the several views, unless context dictates otherwise.

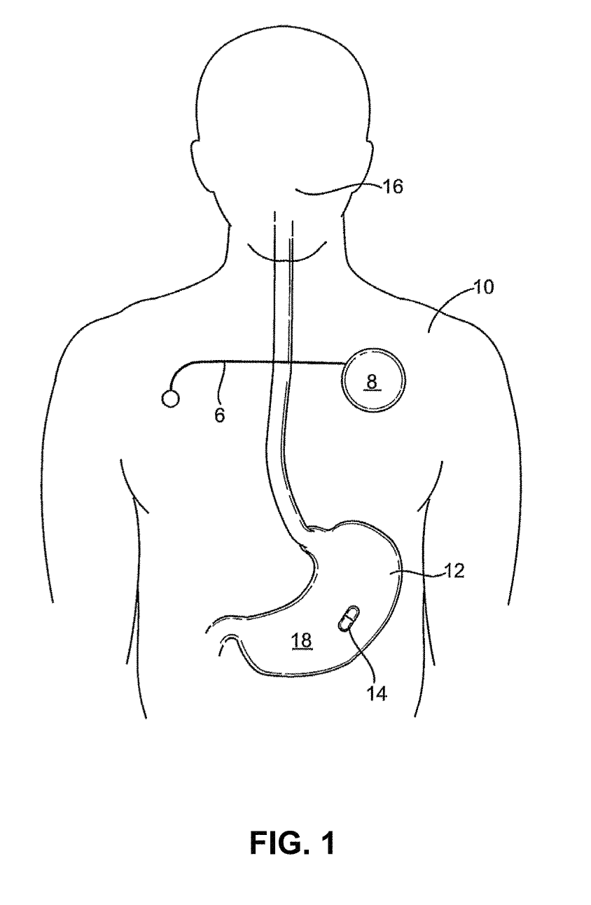

FIG. 1 is a diagrammatic, exemplary representation of a pill/capsule embodiment of the present disclosure, according to one aspect of the present disclosure, in which the composition is configured as an orally ingestible pharmaceutical formulation in the form of a pill or capsule. The stomach 12 of the patient 10 who ingests the composition 14 is shown. This “smart pill” is shown as it has traveled from the mouth 16 to inside 18 the patient's stomach. Upon reaching the stomach, the pill/capsule may undergo a dissolving process with both the mechanical action of the stomach and the various chemical materials in the stomach fluids, such as hydrochloric acid and other digestive agents.

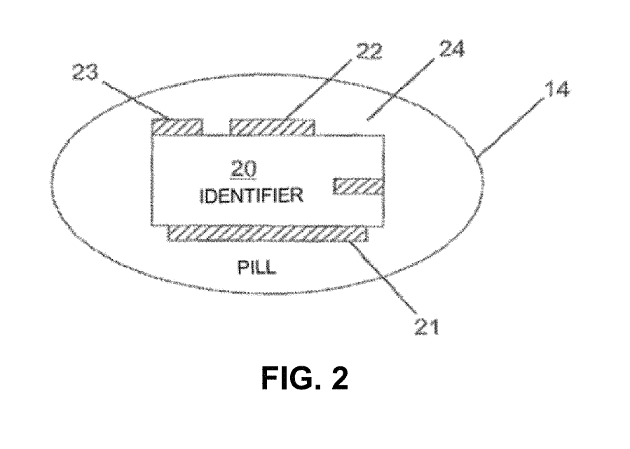

FIG. 2 is a more detailed view of the pill composition shown in FIG. 1. FIG. 2 illustrates an identifier 20 disposed inside a pill 14. Identifier 20 is present as an integrated circuit (IC). The backside (bottom) of circuit 20 may be at least partially coated with a first metal 21, and a portion of the front (top) of circuit 20 may be coated with a different metal 22, allowing circuit 20 to be powered by reverse electrolysis. Also on the top surface may be two transmitter electrodes 23, 24.

When pill 14 is fabricated, the integrated circuit 20 may be surrounded by at least one external layer that may include pharmacologically active and/or inert materials in any combination. The external layer may dissolve in the stomach through a combination of the mechanical action of the stomach and the action of various chemical constituents (e.g., hydrochloric acid) in stomach fluids.

As pill 14 is dissolved, areas of integrated circuit 20 may become exposed to the stomach contents, which for present purposes can be regarded as an electrolyte solution. As dissolution of the pill exposes metal layers 21 and 22 (magnesium metal and copper chloride), power may be supplied to circuit 20, which may begin to operate and continue to operate until metal layers 21 and 22 or the circuit itself are sufficiently dissolved by digestive processes and acids to become non-functional. Eventually, the remains of the chip are excreted from the body.

In an alternative embodiment, the integrated circuit 20 may be attached to, rather than encapsulated in, the pill 14. For instance, circuit 20 might be placed at one end of the pill as the pill is being prepared, in a soluble coating on the surface of the pill, or the like. In embodiments where circuit 20 is wholly or partially exposed, integrated circuit 20 may begin to operate sooner after the pill enters the stomach rather than after the pill dissolves.

In one embodiment, circuit 20 may transmit a signal identifying pill 14. The identifier may indicate the type of active ingredient, brand, etc. and/or dosage of pill 14 and may also provide a lot number, serial number, or similar identifying information that would allow particular pills to be traced, e.g., as reviewed above.

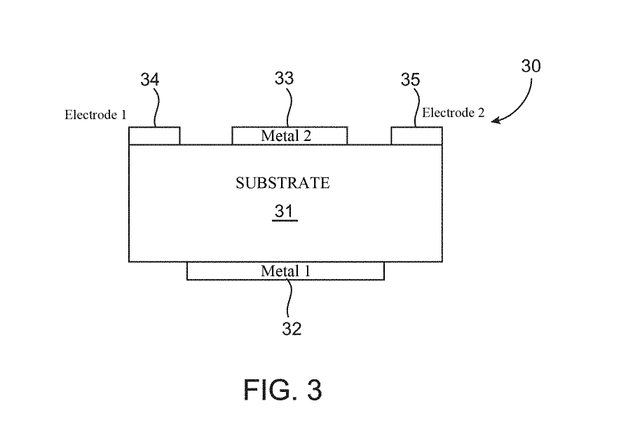

FIG. 3 is a detailed depiction of an embodiment of a signal generation element 30 which labels the pharmaceutical material and is encapsulated in the center of the composition, according to one aspect of the present disclosure. Signal generation element 30 is in the form of IC constructed from a silicon chip where various functional elements, e.g., in the form of one or more layers of circuits, may be disposed on a silicon substrate 31. The chip can be fabricated using standard integrated circuit techniques. An example of such a fabrication approach may be a 0.5 CMOS process made available by AMI Semiconductor in Idaho, USA. Shown on the backside of the substrate, the bottom of the chip 31 may be metal 1 32 which functions as one battery electrode (magnesium metal or copper chloride), and on the topside of the chip may be metal 2 33 which functions as the other battery electrode (copper chloride, or magnesium metal). Also on the top side of the chip 31 may be electrode 1 34 and electrode 2 35, which may constitute a pair of signal-transmission electrodes.

In some cases, dissolution of the electrodes, and thus extinction of the reporting signal, can provide a secondary indication of the full dissolution of the pill and incorporated devices.

A potential applied to the silicon may be a positive voltage on the top surface and a negative voltage on the bottom surface. In this way, the substrate may be essentially at the same potential as the cathode, which can be the ground reference for the circuits, and the top surface, with a SiO₂ insulation layer, may be coupled to a positive voltage, referenced to that ground on the bottom side.

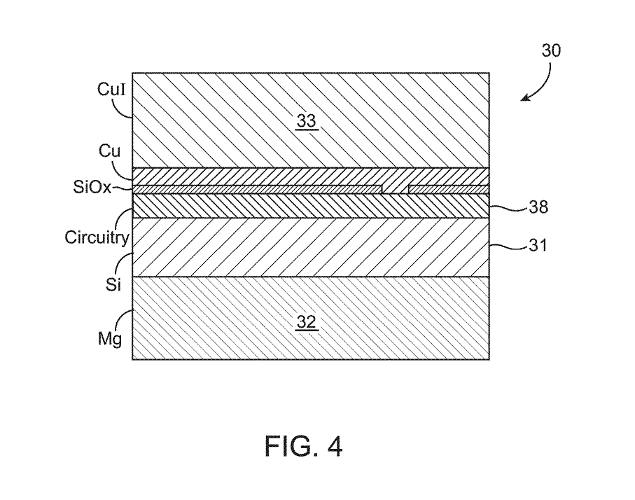

In certain embodiments, the signal generation element may not include antennae and instead uses battery components as antennae, such as shown in FIG. 4. In FIG. 4, signal generation element 30 may include silicon support layer 31 positioned between metal 1 layer 32 and metal 2 layer 33. Also shown is circuitry layer 38. In such embodiments, when a switch on the chip, e.g., in the circuitry layer, is closed, a current may be produced between the two metals of the battery, which is then detected. In certain embodiments, a membrane larger than the chip, which defines a path for the current to travel, may be provided.

### Methods of Making Compositions

A variety of manufacturing protocols may be employed to produce compositions according to the present disclosure. In manufacturing the subject compositions, a signal generation element may be stably associated with the pharmaceutical dosage such that the signal generation element and the dosage do not separate from each other, at least until administered to the subject in need thereof, e.g., by ingestion. The signal generation element may be stably associated with the pharmaceutical carrier/active ingredient component of the composition in a number of different ways.

In some embodiments, where the carrier/active ingredient component is a solid structure, e.g., such as a tablet or pill, the carrier/active ingredient component may be produced in a manner that provides a cavity for the signal generation element. The signal generation element may then be placed into the cavity and the cavity sealed, e.g., with a biocompatible material, to produce the final composition. For example, in certain embodiments, a tablet may be produced with a die that includes a feature which produces a cavity in the resultant compressed tablet. The signal generation element may be placed into the cavity and the cavity sealed to produce the final tablet. In a variation of this embodiment, the tablet may be compressed with a removable element, e.g., in the shape of a rod or other convenient shape. The removable element may then be removed to produce a cavity in the tablet. The signal generation element may be placed into the cavity and the cavity sealed to produce the final tablet. In another variation of this embodiment, a tablet without any cavity is first produced and then a cavity is produced in the tablet, e.g., by laser drilling. The signal generation element is placed into the cavity and the cavity sealed to produce the final tablet.

In some embodiments, a tablet may be produced by combining the signal generation element with subparts of the tablet, where the subparts may be pre-made subparts or manufactured sequentially. For example, in certain embodiments tablets may be produced by first making a bottom half of the tablet, placing the signal generation element on a location of the bottom half of the tablet, and then placing top portion of the tablet over the bottom half and signal generation element to produce the final desired composition.

In some embodiments, a tablet may be produced around a signal generation element such that the signal generation element is located inside of the produced tablet. For example, a signal generation element, which may or may not be encapsulated in a biocompatible compliant material, e.g., gelatin (to protect the signal generation element), may be combined with carrier/active ingredient precursor, e.g., powder, and compressed or molded into a tablet in a manner such that the signal generation element is located at an internal position of the tablet.

The inventors have recognized that it is difficult to combine a pharmaceutical compound with an IEM device to manufacture a stable pharmaceutical product with a reasonable shelf life that meets the FDA requirements and still achieve the functions of the IEM device. For example, tablets may be manufactured by pressing the pharmaceutical compound with a certain pressure, but when an IEM device is combined with the pharmaceutical compound to make the tablets, the pressure used to press the tablets must be carefully tested. Too much pressure would likely damage the IEM device, but if too little pressure is used, the manufactured tablets may not have the desired hardness and other properties to meet the FDA requirements. Further, the conditions of the manufacturing process may vary depending on the specific compositions used, such as the active ingredient, the elements/compositions of the IEM device, and the amounts thereof, which may also affect the properties of the manufactured pharmaceutical product, such as tablets. The inventors have surprisingly discovered that the compositions of the present disclosure, for example, when manufactured as described in greater detail below, may meet the desired requirements while still achieving the desired functions of the IEM device.

Accordingly, the present disclosure provides a unique composition of matter comprising the combination of the IEM electronic circuitry comprising battery forming materials and specific formulations of active ingredient to confirm the delivery of the specific formulations of active ingredient (as described herein). The compositions provided herein overcome the unpredictable nature (e.g., impact on functionality, shelf-life, structural stability, chemical stability, etc.) of combining various metals and salts with the specific formulations of active ingredient (as described herein). to provide an electronic IEM delivery system that generates its own electrical power from a partial energy source comprised of dissimilar materials when exposed with the bodily fluids of a patient during the oral administration of the specific formulations of active ingredient (as described herein).

### EXAMPLES

### Example 1 Manufacturing of Power Source and IEM

According to one aspect of the present disclosure, a partial power source may be manufactured as described in detail herein.

In some embodiments, the anode may be made of metals including but not limited to magnesium, zinc, sodium, lithium, iron, and alloys thereof. Certain high energy anode material such as sodium, lithium, and other alkali metals may be unstable in their pure form in the presence of water or oxygen. These may however be used in an aqueous environment if stabilized. One example of this stabilization is the so-called “protected lithium anode” developed by Polyplus Corporation (Berkeley, Calif.), where a polymer film is deposited on the surface of lithium metal to protect it from rapid oxidation and allow its use in aqueous environment or air ambient. In other embodiments, the anode may include intercalation compounds such as graphite with lithium, potassium, calcium, sodium, and/or magnesium. The anode including the intercalation compounds may have greater surface areas, which may generate stronger signals. Such an intercalation compound can include graphite intercalated with an element selected from the group consisting of lithium, potassium, calcium, sodium, magnesium, and combinations thereof.

In some embodiments, the cathode may include copper salts of iodide, chloride, bromide, sulfate, formate, or any other suitable anions. In other embodiments, the cathode may include Fe³⁺ salts of orthophosphate, pyrophosphate, or any other suitable anions. In some other embodiments, the cathode may include vanadium oxide and/or manganese oxide. In some other embodiments, dissolved oxygen can also serve as a cathode. In this case, the dissolved oxygen in the bodily fluids may be reduced to OH— at a suitable catalytic surface such as platinum or gold or other catalytic surfaces. Also of interest may be dissolved hydrogen in a hydrogen reduction reaction.

A semiconductor substrate may be provided as a chassis which components of the IEM are attached to, deposited upon, and/or secured to. The substrate may be made of silicon. The cathode material may be physically associated with the substrate (e.g., on one side). The cathode material may be chemically deposited on, evaporated onto, secured to, or built-up on the substrate all of which may be referred to herein as “deposit” with respect to the substrate. The cathode material may be deposited on one side of the substrate. The cathode material may be deposited by physical vapor deposition, electrodeposition, or plasma deposition, among other protocols. The cathode material may be from about 0.05 to about 500 μm thick, such as from about 5 to about 100 μm thick. The shape may be controlled by shadow mask deposition, or photolithography and etching. Additionally, there may be more than one electrically unique region on the substrate where the cathode material may be deposited, as desired.

At a different side, which may be the opposite side to the side where the cathode material is deposited, the anode material may be deposited. The different side selected may be the side next to the side selected for the cathode material. The scope of the present disclosure is not limited by the side selected, and the term “different side” can mean any of the multiple sides that are different from the first selected side. Furthermore, the shape of the deposited material(s) may be any geometrically suitable shape. The materials are selected such that they may produce a voltage potential difference when the power source is in contact with conducting liquid, such as body fluids. As indicated above with respect to the cathode material, the anode material may be chemically deposited on, evaporated onto, secured to, or built-up on the substrate. Also, an adhesion layer may be necessary to help the anode material (as well as the cathode material when needed) to adhere to the substrate to provide better electrode contact between the substrate and the electrode material. Typical adhesion layers for the anode material may be Au, Ti, TiW, Cr or similar material. The adhesive layer may have a thickness from 50 Å to 100 Å and up to 1 μm (e.g., about 50 Å to about 1 μm, about 100 Å to about 1 μm, or about 50 Å to about 100 Å). The anode material and the adhesion layer may be deposited by physical vapor deposition, electrodeposition or plasma deposition. The anode material may be from about 0.05 to about 500 μm thick, such as from about 5 to about 100 μm thick. However, the scope of the present disclosure is not limited by the thickness of any of the materials nor by the type of process used to deposit or secure the materials to the substrate.

According to the disclosure set forth, when used with ingestible active ingredient IEM tablets manufactured as described below, the electrode materials are magnesium metal and copper chloride. That is, the anode comprises magnesium metal, and the cathode comprises copper chloride.

In some embodiments, the power source in each active ingredient IEM tablet, manufactured as described below, may include about 0.9 mg of Si, 0.2 mg of Cu, and 0.01 mg of Mg. There is a thick (˜1 um) layer of gold under the CuCl to increase the surface roughness. The amounts of the materials may be sufficient for generating enough power for the IEM to have a communication time of at least about 10 minutes. A target communication time may be about 1.5 hours. The power source in each active ingredient IEM tablet may include at least 0.09 mg of Si, 0.02 mg of Cu, and 0.001 mg of Mg. The greater surface areas the electrodes may have, the more power and the stronger signals the IEM may generate, and at the same time, the more materials the power source may have. However, the quantities of the materials used may have to meet the requirements set forth by FDA with respect to the specific elements. Therefore, for example, the maximum amounts of Si, Cu, and Mg, respectively, in each tablet may not exceed the maximum amounts of Si, Cu, and Mg, respectively, as set forth by FDA.

In certain aspects, these two electrode materials may be shielded from the surrounding environment by an additional layer of material. Accordingly, when the shield is dissolved and the two dissimilar materials (magnesium metal and copper chloride) are exposed to the target site, a voltage potential is generated.

Other components of the IEM may be provided as described above.

### Example 2 Manufacturing of Active Ingredient IEM Tablets

According to one aspect of the present disclosure, active ingredient IEM tablets may be manufactured using the IEM manufactured in Example 1 and using the processes described in U.S. Pat. No. 8,784,308. An illustration process is described below.

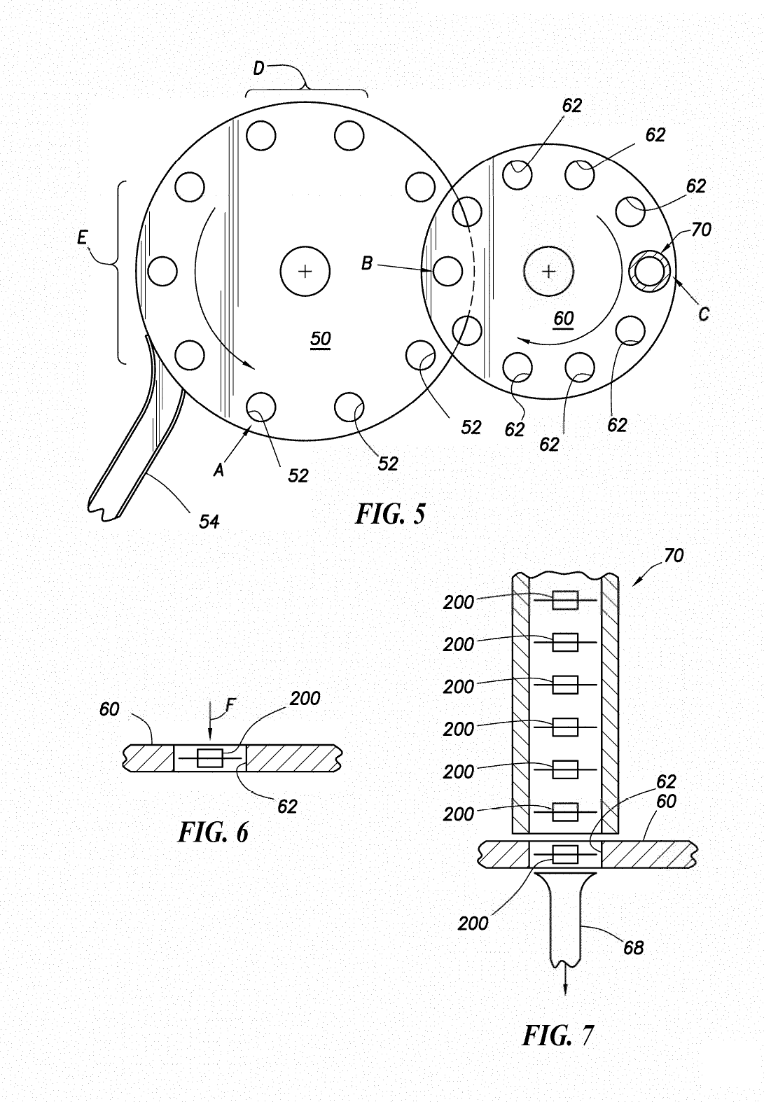

As in FIGS. 5-7, a tablet press 50 is shown. The press 50 may rotate in a counter-clockwise direction as shown. The press 50 may include die cavity or punch cavity 52 and an ejection tray 54. Starting at position A, as shown, the pharmaceutical product, such as e.g., aripiprazole, risperidone, quetiapine, or brexpiprazole, may be deposited in the cavity 52. The press 50 may rotate to position B, which may be positioned below a transfer wheel 60. The wheel 60 may include several openings 62. As the wheel 60 passes position C, each opening 62 may pass under a feeder 70, as shown in FIG. 7.

The feeder 70 may contain marker devices 200. The device 200 may be an IEM that is activated upon contact with a conducting fluid, manufactured as described above in Example 1. The scope of the present disclosure is not limited by the environment or type of the conducting fluid. Once ingested, the device 200 may come into contact with a conducting fluid, such as stomach fluids, and the device 200 may be activated. Referring to the instance where the device 200 is used with the product that is ingested by the living organism, when the product that includes the device 200 is taken or ingested, the device 200 may come into contact with the conducting liquid of the body, a voltage potential may be created, and the device 200 may be activated. A portion of the power source may be provided by the device 200, such as the electrode materials as described above, while another portion of the power source may be provided by the conducting fluid.

Referring again to FIGS. 5 and 6, each time an opening 62 passes under the feeder 70, one of the devices 200 may be dropped into the opening 62 directly under the feeder 70. As shown in FIG. 6, a force “F” is shown to assist the movement of the device 200 from the feeder 70 into the opening 62. The force may be provided by the use of a vacuum through a suction tube 68. In accordance with other aspects of the present disclosure, the force may be provided by a spring, an air burst, or an ejection pin in addition to gravity. The wheel 60 may rotate to position B. At position B, the device 200 located in the opening 62 may be dropped into the cavity 52 of the press 50. The press 50 may rotate to the position D where additional pharmaceutical product may be deposited into the cavity 52 on top of the device 200. The press 50 may continue to move in the counter-clockwise direction and at position E, the content of the cavity 52 may be pressed under high pressure to form a tablet with the device 200 inside. The completed tablet may be ejected and moved to a collection point through the ejection tray 54 for further processing, such as coating layers as needed.

Referring now to FIG. 8, a feeder assembly 72 is shown as alternative embodiment and in accordance with another aspect of the present disclosure. The feeder assembly 72 can be used in place of the feeder 70 of the FIG. 5. The feeder assembly 72 may include a plurality of supporting fingers 74 that hold each device 200 in position. The fingers 74 may be connected to a belt 76. The fingers 74 may lower the device 200 toward the wheel 60 of FIG. 5. When the fingers 74 may reach the lower portion near the wheel 60, the fingers 74 may move apart and drop the device 200 into the opening 62 of the wheel 60.

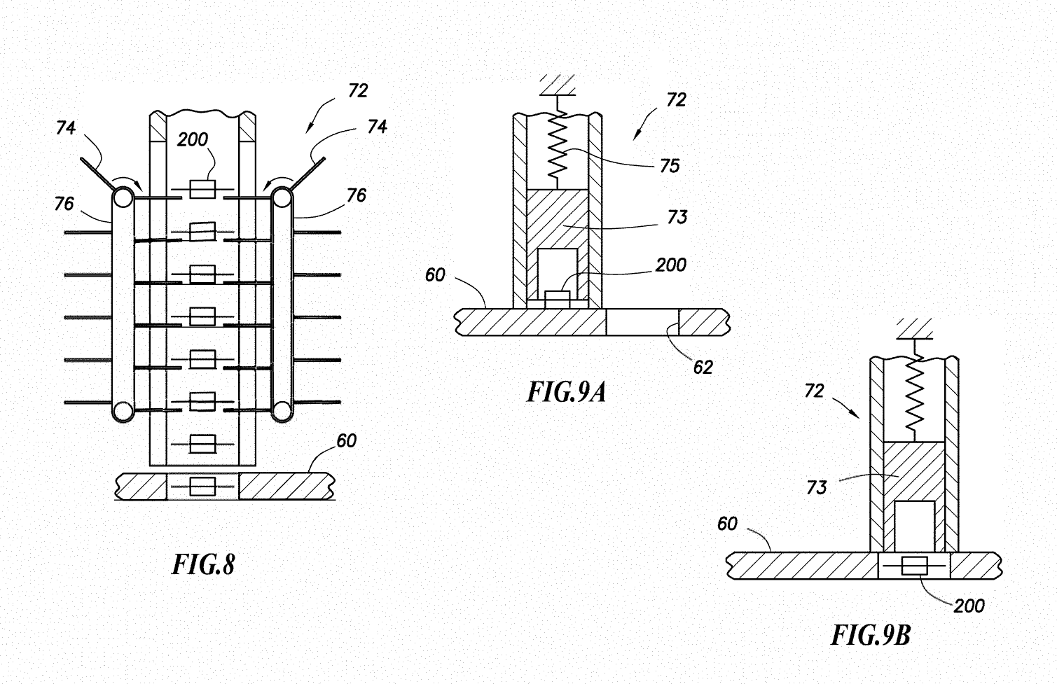

Referring now to FIG. 9A and FIG. 9B, in accordance with another aspect of the present disclosure, the feeder assembly 72 may include an ejector 73 with a spring 75. As the opening 62 moves under the feeder assembly 72, the ejector 73 may push the device 200 into the opening 62 of the wheel 60.

According to one aspect of the present disclosure, the active ingredient IEM tablets are manufactured according to Table 1 using a Modified Rectangle Tablet Press Tool Set (4.5×8 mm) and 6.0 mm, 7.8 mm, and 9.0 mm Diameter Concave Beveled Edge Tablet Press Tool Sets, by Elizabeth Carbide Aripiprazole*Die Co, with 0.25 mg, 0.5 mg, 1 mg, 2 mg 3 mg, 4 mg, 5 mg, 15 mg, 20 mg, 25 mg, 50 mg, 30 mg, 100 mg, 200 mg, 300 mg, and 400 mg of the active ingredient, respectively.

TABLE 1
ACTIVE INGREDIENT IEM Tablets

| Tablets | Description | Tool Set |
|---|---|---|
| ACTIVE | ACTIVE INGREDIENT IEM | Modified Rectangle |
| INGREDIENT IEM | Tablet for 2 mg and 5 mg | Tablet Press |
| Tablet 2-5 mg | Placebo Modified Rectangle | Tool Set |
| Placebo | Shape 4.5 × 8 mm | (4.5 × 8 mm) |
| ACTIVE | ACTIVE INGREDIENT IEM | 6.0 mm Diameter |
| INGREDIENT IEM | Tablet for 15 mg Placebo | Tablet Press |
| Tablet 15 mg | 6.0 mm Round Shape | Tool Set |
| Placebo | | |
| ACTIVE | ACTIVE INGREDIENT IEM | 7.8 mm Diameter |
| INGREDIENT IEM | Tablet for 20 mg Placebo | Tablet Press |
| Tablet 20 mg | 7.8 mm Round Shape | Tool Set |
| Placebo | | |
| ACTIVE | ACTIVE INGREDIENT IEM | 9.0 mm Diameter |
| INGREDIENT IEM | Tablet for 30 mg Placebo | Tablet Press |
| Tablet 30 mg | 9.0 mm Round Shape | Tool Set |
| Placebo | | |

Lots of the ACTIVE INGREDIENT IEM tablets in Table 1 were manufactured according to Table 2.

TABLE 2
Lots of ACTIVE INGREDIENT IEM Tablets

| | | | Test Samples (n) | | | |
|---|---|---|---|---|---|---|
| Digital Tablet | | | | In | Lot | |
| Type | Tools | Tablet Lots | A/S* | Process | release** | Total |
| ACTIVE | Modified | 1 (Low hardness) | NA | 100 | NA | 100 |
| INGREDIENT | Rectangle Tablet | 5 (High hardness) | NA | 100 | NA | 100 |
| IEM Tablet 2- | Press Tool Set | 2 (Target hardness) | 40/ | 30/Lot | 220/Lot | 290/Lot |
| 5 mg Placebo | (4.5 × 8 mm) | 3 (Target hardness) | Lot | | | |
| | | 4 (Target hardness) | | | | |
| ACTIVE | 6.0 mm Diameter | 6 (Low hardness) | NA | 100 | NA | 100 |
| INGREDIENT | Tablet Press | 10 (High hardness) | NA | 100 | NA | 100 |
| IEM Tablet 15 mg | Tool Set | 7 (Target hardness) | 40/ | 30/Lot | 220/Lot | 290/Lot |
| Placebo | | 8 (Target hardness) | Lot | | | |
| | | 9 (Target hardness) | | | | |
| ACTIVE | 7.8 mm Diameter | 11 (Low hardness) | NA | 70 | NA | 70 |
| INGREDIENT | Tablet Press | 15 (High hardness) | NA | 70 | NA | 70 |
| IEM Tablet 20 mg | Tool Set | 12 (Target hardness) | 40/ | 30/Lot | 180/Lot | 250/Lot |
| Placebo | | | Lot | | | |
| | | 13 (Target hardness) | | | | |
| | | 14 (Target hardness) | | | | |
| ACTIVE | 9.0 mm Diameter | 16 (Low hardness) | NA | 60 | NA | 60 |
| INGREDIENT | Tablet Press | 20 (High hardness) | NA | 60 | NA | 60 |
| IEM Tablet 30 mg | Tool Set | 17 (Target hardness) | 40/ | 30/Lot | 180/Lot | 250/Lot |
| Placebo | | | Lot | | | |
| | | 18 (Target hardness) | | | | |
| | | 19 (Target hardness) | | | | |

*A/S (Animal Study): Each lot had a specific IEM TID. Samples were taken for an animal study
**Additional samples per lot were for analytical retains.

Low hardness means low compression force was used. High hardness means high compression force was used. Target hardness means target compression force was used. The compression force and dwell time parameters used for manufacturing the active ingredient IEM tablets in Tables 1 and 2 are shown in Table 3.

TABLE 3
Compression Force and Dwell Time Parameters

| | | Low | | High | | | |
|---|---|---|---|---|---|---|---|
| | | Comp | Target | Comp | | | |
| | | Force | Comp | Force | | | |
| | | Used | Force Used | Used | Target | Target | Dwell |
| Digital Tablet Type | Tablet Lot | (lbf) | (lbf) | (lbf) | (SCU) | (kp) | Time |
| ACTIVE INGREDIENT | 1 (Low | 7000 | | | 7.0 | 5.0 | 4 sec |
| IEM Tablet 2-5 mg | hardness) | | | | | | |
| Placebo | 5 (High | | | 16500 | 13.0 | 9.3 | |
| | hardness) | | | | | | |
| | 2 (Target | | 13000 | | 10.0 | 7.1 | |
| | hardness) | | | | | | |
| | 3 (Target | | 13000 | | 10.0 | 7.1 | |
| | hardness) | | | | | | |
| | 4 (Target | | 13000 | | 10.0 | 7.1 | |
| | hardness) | | | | | | |
| ACTIVE INGREDIENT | 6 (Low | 8000 | | | 7.0 | 5.0 | |
| IEM Tablet 15 mg | hardness) | | | | | | |
| Placebo | 10 (High | | | 16500 | 13.0 | 9.3 | |
| | hardness) | | | | | | |
| | 7 (Target | | | | 10.0 | 7.1 | |
| | hardness) | | | | | | |
| | 8 (Target | | | 13000 | 10.0 | 7.1 | |
| | hardness) | | | | | | |
| | 9 (Target | | | 13000-14000 | 10.0 | 7.1 | |
| | hardness) | | | | | | |
| ACTIVE INGREDIENT | 11 (Low | 10000 | | | 7.0 | 5.0 | |
| IEM Tablet 20 mg | hardness) | | | | | | |
| Placebo | 15 (High | | | 16200 | 10.0 | 7.1 | |
| | hardness) | | | | | | |
| | 12 (Target | | 14000 | | 10.0 | 7.1 | |
| | hardness) | | | | | | |
| | 13 (Target | | 14000 | | 10.0 | 7.1 | |
| | hardness) | | | | | | |
| | 14 (Target | | 14000 | | 10.0 | 7.1 | |
| | hardness) | | | | | | |
| ACTIVE INGREDIENT | 16 (Low | 10300 | | | 7.0 | 5.0 | |
| IEM Tablet 30 mg | hardness) | | | | | | |
| Placebo | 20 (High | | | 16000 | 13.0 | 9.3 | |
| | hardness) | | | | | | |
| | 17 (Target | | | 13000 | 10.0 | 7.1 | |
| | hardness) | | | | | | |
| | 18 (Target | | | 13000 | 10.0 | 7.1 | |
| | hardness) | | | | | | |
| | 19 (Target | | | 13000-13500 | 10.0 | 7.1 | |
| | hardness) | | | | | | |

### Example 3 In-Process Testing

In-process sampling was performed at start of the lot (0% of the total defined number of tablets in a lot), midpoint of the lot (40% per DHR), and end of the lot (100% of the total defined number of tablets in a lot). N is the sample size. The sample size for each Lot of target hardness was 10 tablets at startup, additional 10 tablets at midpoint and 10 more at the end of process for each Lot. The sample size for each high or low hardness Lot was 10 tablets. The sampled tablets were measured according to Table 4.

TABLE 4
In-process testing data

| | | | Actual Tablet | Actual Tablet | Actual Tablet |
|---|---|---|---|---|---|
| | | | weight (mg), | Thickness (mm), | Hardness (kp), |
| Tablet | | Compression | N = 10 | N = 10 | N = 10 |
| shape/Size | | force (lbs) @ | High Low, | High Low, | High, Low, |
| (mm) | Tablet Lot | 4 s dwell time | N = 30 Target | N = 30 Target | N = 30 Target |
| Modified | 1 (Low hardness) | 7000 | 98.3 ± 0.7 | 2.79 ± 0.02 | 5.8 ± 0.3 |
| Rectangle | 2 (Target hardness) | 13000 | 99.8 ± 2.2 | 2.70 ± 0.04 | 7.6 ± 0.7 |
| 4.5 × 8.0 | 3 (Target hardness) | | 99.1 ± 2.3 | 2.65 ± 0.04 | 7.7 ± 0.8 |
| | 4 (Target hardness) | | 98.5 ± 2.2 | 2.63 ± 0.05 | 7.9 ± 0.8 |
| | 5 (High hardness) | 16500 | 99.7 ± 1.1 | 2.61 ± 0.02 | 8.9 ± 0.6 |
| Round | 6 (Low hardness) | 8000 | 100.9 ± 1.5 | 3.00 ± 0.03 | 5.0 ± 0.5 |
| 6.0 mm | 7 (Target hardness) | 13000 | 98.1 ± 3.1 | 2.80 ± 0.06 | 7.1 ± 1.0 |
| | 8 (Target hardness) | | 99.2 ± 2.5 | 2.80 ± 0.06 | 7.1 ± 1.0 |
| | 9 (Target hardness) | | 101.8 ± 2.3 | 2.92 ± 0.06 | 6.4 ± 0.8 |
| | 10 (High hardness) | 16500 | 98.3 ± 1.9 | 2.76 ± 0.03 | 8.2 ± 0.9 |
| Round | 11 (Low hardness) | 10000 | 191.8 ± 2.9 | 3.57 ± 0.04 | 5.5 ± 0.4 |
| 7.8 mm | 12 (Target hardness) | 14000 | 194.9 ± 4.2 | 3.50 ± 0.06 | 7.0 ± 0.6 |
| | 13 (Target hardness) | | 194.1 ± 4.7 | 3.51 ± 0.08 | 7.4 ± 0.7 |
| | 14 (Target hardness) | | 192.1 ± 3.0 | 3.49 ± 0.04 | 6.8 ± 0.6 |
| | 15 (High hardness) | 16200 | 190.8 ± 2.5 | 3.39 ± 0.04 | 8.2 ± 0.5 |
| Round | 16 (Low hardness) | 10300 | 290.8 ± 5.6 | 4.19 ± 0.05 | 5.3 ± 0.7 |
| 9.0 mm | 17 (Target hardness) | 13000 | 292.3 ± 5.0 | 4.15 ± 0.05 | 6.6 ± 0.6 |
| | 18 (Target hardness) | | 291.5 ± 4.7 | 4.10 ± 0.05 | 6.8 ± 0.8 |
| | 19 (Target hardness) | | 291.8 ± 5.0 | 4.10 ± 0.07 | 6.7 ± 0.7 |
| | 20 (High hardness) | 16000 | 288.3 ± 5.8 | 3.96 ± 0.08 | 7.9 ± 0.9 |

### Example 4 Release Testing

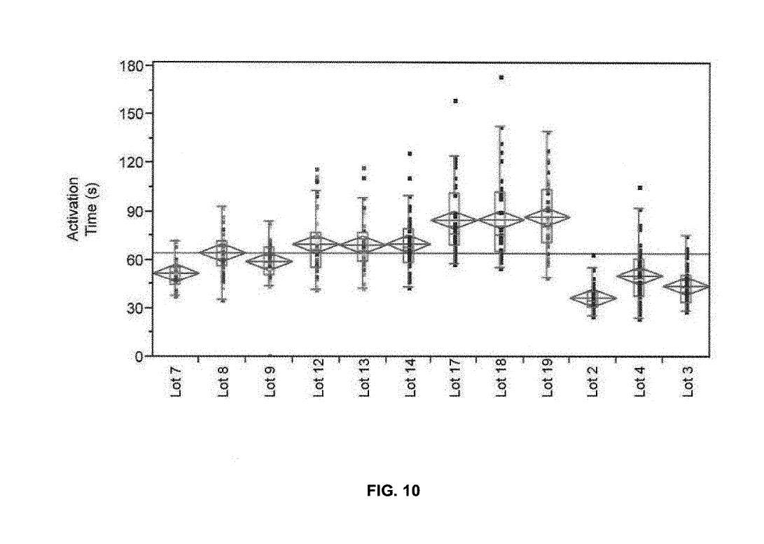

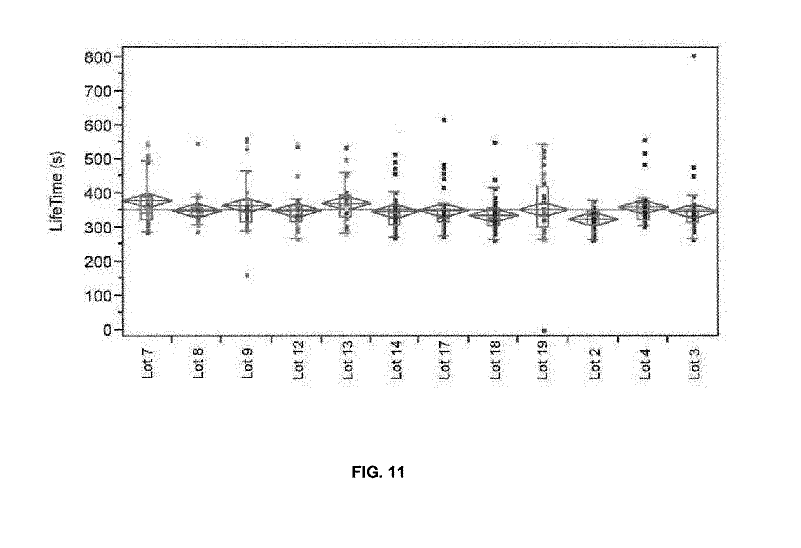

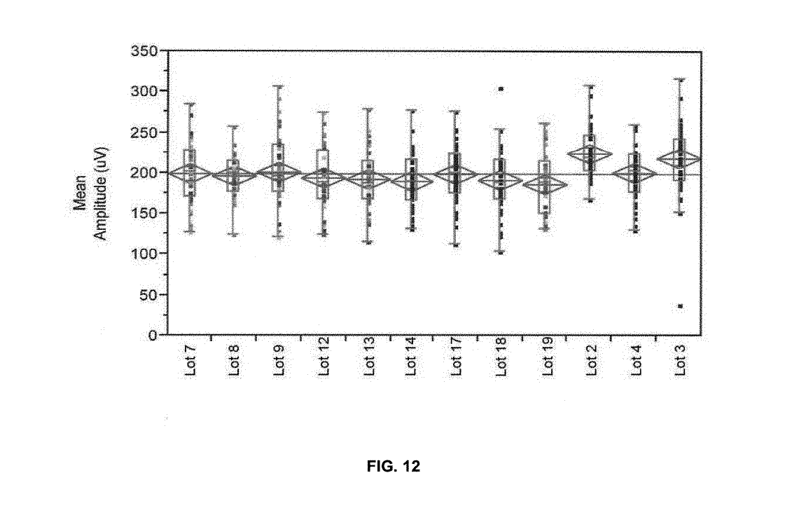

After the manufacturing of all the tablets was completed, the retained tablets were sampled for release testing. The release testing data is shown in Table 5 and FIGS. 10-12.

TABLE 5
Release testing data

| | | Compression | | | | | |
|---|---|---|---|---|---|---|---|
| | | force | Tablet | Tablet | | | |
| Tablet | | (lbs) # 4 s | weight | Thickness | Tablet | | Disintegration |
| shape/Size | | dwell | (mg), | (mm), | Hardness | Friability | Time |
| (mm) | Tablet Lot | time | N = 15 | N = 15 | (kp), N = 15 | (%) ≥6.5 g | (min),* N = 6 |
| Modified | 1 (Low | 7000 | 97.3 ± 1.5 | 2.77 ± 0.05 | 4.2 ± 0.4 | 0.14 | 1 |
| Rectangle | hardness) | | | | | | |
| 4.5 × 8.0 | 2 (Target | 13000 | 97.3 ± 1.6 | 2.77 ± 0.06 | 5.4 ± 0.5 | 0.08 | 2 |
| | hardness) | | | | | | |
| | 3 (Target | | 99.5 ± 2.4 | 2.65 ± 0.04 | 5.9 ± 0.7 | 0.10 | 2 |
| | hardness) | | | | | | |
| | 4 (Target | | 98.7 ± 3.8 | 2.63 ± 0.06 | 5.8 ± 0.9 | 0.11 | 2 |
| | hardness) | | | | | | |
| | 5 (High | 16500 | 97.9 ± 2.0 | 2.58 ± 0.04 | 7.4 ± 0.9 | 0.07 | 2 |
| | hardness) | | | | | | |
| Round | 6 (Low | 8000 | 100.2 ± 2.0 | 2.98 ± 0.04 | 4.2 ± 0.5 | 0.05 | 1 |
| 6.0 mm | hardness) | | | | | | |
| | 7 (Target | 13000 | 103.0 ± 2.2 | 2.96 ± 0.06 | 5.1 ± 0.6 | 0.06 | 2 |
| | hardness) | | | | | | |
| | 8 (Target | | 99.9 ± 1.9 | 2.87 ± 0.05 | 5.7 ± 0.6 | 0.06 | 3 |
| | hardness) | | | | | | |
| | 9 (Target | | 99.6 ± 2.7 | 2.83 ± 0.06 | 6.0 ± 0.8 | 0.06 | 2 |
| | hardness) | | | | | | |
| | 10 (High | 16500 | 98.2 ± 2.0 | 2.78 ± 0.04 | 6.8 ± 0.7 | 0.07 | 2 |
| | hardness) | | | | | | |
| Round | 11 (Low | 10000 | 190.7 ± 3.5 | 3.58 ± 0.03 | 4.8 ± 0.6 | 0.10 | 3 |
| 7.8 mm | hardness) | | | | | | |
| | 12 (Target | 14000 | 194.8 ± 5.3 | 3.60 ± 0.10 | 5.1 ± 0.6 | 0.08 | 3 |
| | hardness) | | | | | | |
| | 13 (Target | | 192.3 ± 4.8 | 3.51 ± 0.06 | 6.2 ± 0.8 | 0.07 | 3 |
| | hardness) | | | | | | |
| | 14 (Target | | 193.9 ± 3.9 | 3.55 ± 0.05 | 5.6 ± 0.8 | 0.09 | 4 |
| | hardness) | | | | | | |
| | 15 (High | 16200 | 190.5 ± 3.4 | 3.40 ± 0.03 | 7.3 ± 0.7 | 0.07 | 3 |
| | hardness) | | | | | | |
| Round | 16 (Low | 10300 | 294.9 ± 4.5 | 4.25 ± 0.05 | 5.4 ± 0.8 | 0.13 | 4 |
| 9.0 mm | hardness) | | | | | | |
| | 17 (Target | 13000 | 296.1 ± 5.7 | 4.24 ± 0.07 | 5.2 ± 0.6 | 0.11 | 3 |
| | hardness) | | | | | | |
| | 18 (Target | | 290.3 ± 4.9 | 4.15 ± 0.07 | 5.1 ± 0.6 | 0.11 | 3 |
| | hardness) | | | | | | |
| | 19 (Target | | 291.2 ± 2.1 | 4.12 ± 0.03 | 6.1 ± 0.6 | 0.10 | 3 |
| | hardness) | | | | | | |
| | 20 (High | 16000 | 293.2 ± 5.0 | 3.99 ± 0.05 | 9.2 ± 1.0 | 0.08 | 4 |
| | hardness) | | | | | | |

*Disintegration time is rounded up to nearest minute
Disintegration time for Modified Rectangle & 6 mm tablets is expressed as the maximum of 6 tablets
Disintegration time for 7.8 & 9 mm tablets is expressed as the average of 6 tablets

The disintegration time, as shown in Table 5, was tested in distilled water at 37 C° as the test medium. Activation Time, Life Time, and Amplitude data, as shown in FIGS. 10-12, was tested in buffered saline solution with 1% w/v Triton X-100.

Activation Time was measured from the moment of complete immersion of the tablet in the test medium to the 5th broadcast of the “defined pill ID” by the tested tablet. Life Time was measured from the Activation Time to the last broadcast of the defined pill ID.

Each of the Activation Time, Life Time, and Amplitude data, as shown in FIGS. 10-12, indicates that the tablets produced for each of the three target hardness lots for each tablet shape are comparable. Furthermore, the data indicates that the process of manufacturing the IEM tablets as described above is capable of running consistently across multiple batches.

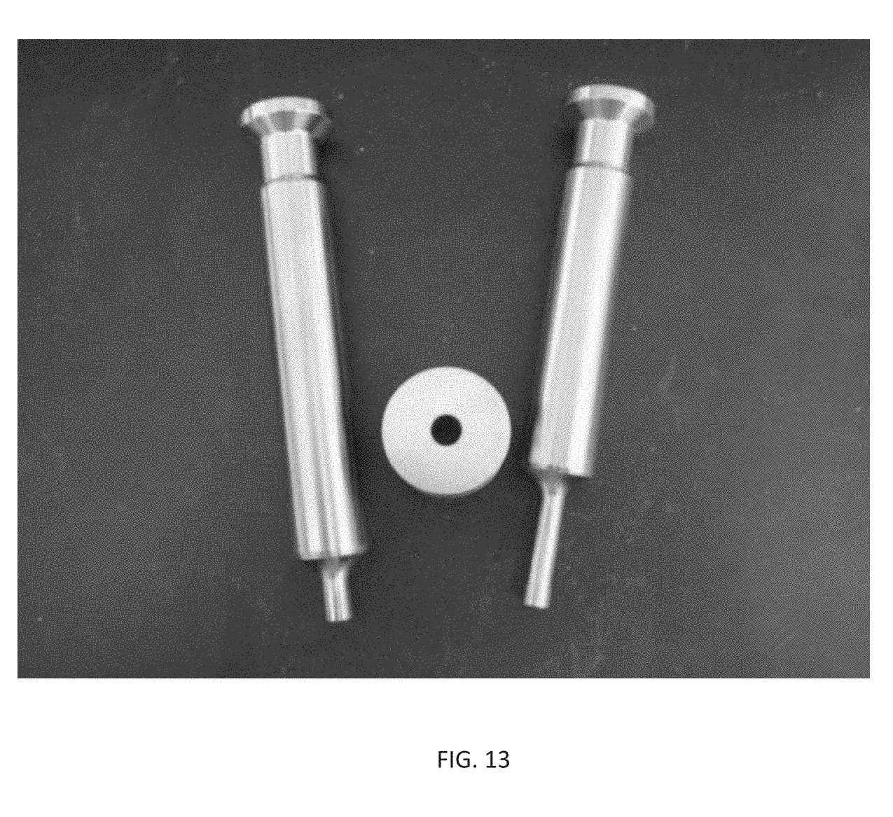

FIG. 13 shows a concave beveled edge tablet press tool set, according to one aspect of the present disclosure.

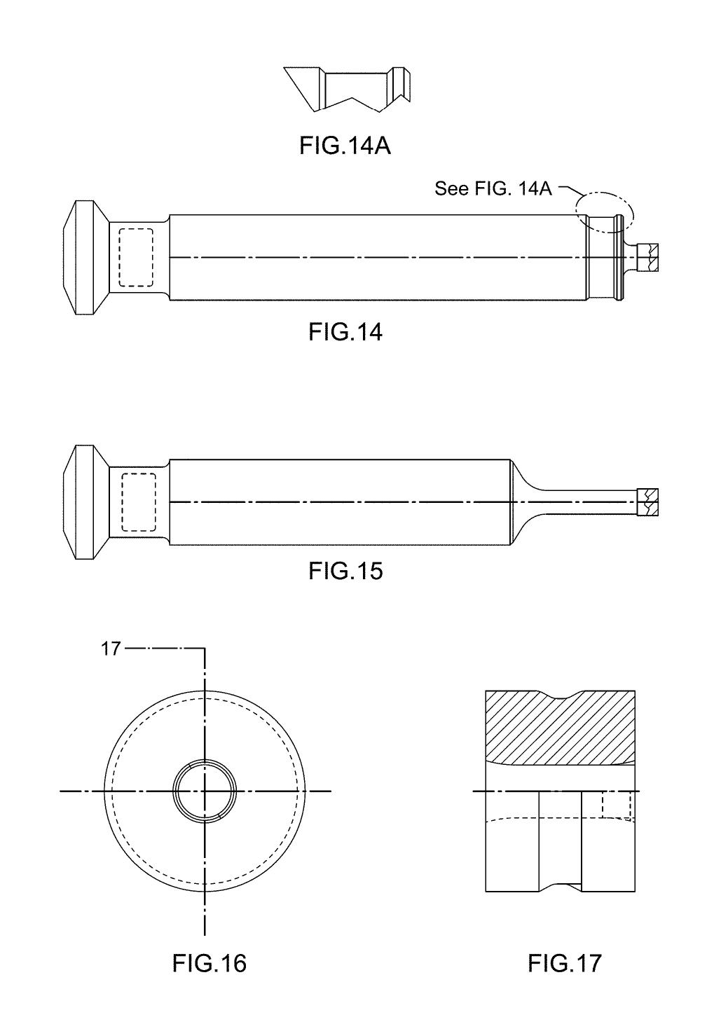

FIG. 14 shows an upper tablet press punch, according to one aspect of the present disclosure.

FIG. 14A is a detail view of the upper tablet press punch shown in FIG. 14, according to one aspect of the present disclosure.

FIG. 15 shows a lower tablet press punch, according to one aspect of the present disclosure.

FIG. 16 shows a plan view of a tablet press die, according to one aspect of the present disclosure.

FIG. 17 is partial section view taken along section line 17 of the tablet press die shown in FIG. 16, according to one aspect of the present disclosure.

In one aspect, the example tool and die set shown in FIGS. 13-17 may be sized and configured to make a 6.0 mm tablets as described herein. In another aspect, the example tool and die set shown in FIGS. 13-17 may be sized and configured to make a 7.8 mm tablets as described herein. In yet another aspect, the example tool and die set shown in FIGS. 13-17 may be sized and configured to make a 9.0 mm tablets as described herein.

It is to be understood that this disclosure is not limited to particular embodiments described, as such may vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

1. A composition of matter for the ingestible administration of aripiprazole, the composition comprising:
aripiprazole;
a metal selected from the group consisting of magnesium, zinc, sodium, lithium, iron, or alloys thereof, or combinations thereof; and
a copper salt selected from the group consisting of copper iodide, copper chloride, copper bromide, copper sulfate, copper formate, or combinations thereof.

2. The composition of claim 1, further comprising an iron salt selected from the group consisting of iron(III) phosphate, iron(III) pyrophosphate, and combinations thereof.

3. The composition of claim 1, further comprising an element selected from the group consisting of Au, Ti, TiW, or Cr, or combinations thereof.

4. The composition of claim 3, comprising an adhesion layer comprising Au, Ti, TiW, or Cr, wherein the adhesive layer has a thickness from 50 Å to 100 Å and up to 1 μm.

5. The composition of claim 1, further comprising silicon.

6. The composition of claim 5, further comprising 0.09-0.9 mg of Si.

7. The composition of claim 1, wherein the copper salt comprises 0.02-0.2 mg of Cu.

8. The composition of claim 1, wherein the metal comprises 0.001-0.01 mg of Mg.

9. The composition of claim 1, further comprising oxygen dissolved in a conducting fluid and reduced to OH— at a catalytic surface.

10. The composition of claim 1, further comprising dissolved hydrogen in a hydrogen reduction reaction.

11. The composition of claim 1, further comprising an intercalation compound.

12. The composition of claim 11, wherein the intercalation compound comprises graphite intercalated with an element selected from the group consisting of lithium, potassium, calcium, sodium, magnesium, and combinations thereof.

13. The composition of claim 1, further comprising vanadium oxide or manganese oxide.

14. The composition of claim 1, comprising 2-30 mg of aripiprazole.

15. The composition of claim 1, comprising 2 mg aripiprazole.

16. The composition of claim 1, comprising 5 mg aripiprazole.

17. The composition of claim 1, comprising 10 mg aripiprazole.

18. The composition of claim 1, comprising 15 mg aripiprazole.

19. The composition of any of claims 15, 16, and 18, having a weight of 94.1-104 mg.

20. The composition of any of claims 15, 16, and 18, having a weight of 99 mg.

21. The composition of any of claims 15, 16, and 18, having a disintegration time of not more than 5 minutes.

22. The composition of claim 1, comprising 20 mg aripiprazole.

23. The composition of claim 22, having a weight of 184.3-203.7 mg.

24. The composition of claim 22, having a weight of 194 mg.

25. The composition of claim 1, comprising 30 mg aripiprazole.

26. The composition of claim 25, having a weight of 274.6-303.5 mg.

27. The composition of claim 25, having a weight of 289 mg.

28. The composition of claim 22 or 25, having a disintegration time of not more than 7 minutes.

29. The composition of claim 1, wherein the composition is in the form of a tablet.

30. The composition of claim 29, wherein the tablet has a rectangular shape.

31. The composition of claim 30, wherein the tablet has a size of 4.5 mm×8 mm.

32. The composition of claim 29, wherein the tablet has a round shape.

33. The composition of claim 32, wherein the tablet has a diameter of 6-9 mm.

34. The composition of claim 32, wherein the tablet has a diameter of 6 mm.

35. The composition of claim 32, wherein the tablet has a diameter of 7.8 mm.

36. The composition of claim 32, wherein the tablet has a diameter of 9 mm.

37. The composition of claim 29, wherein the tablet has a thickness of 2.59-4.24 mm.

38. The composition of claim 29, wherein the tablet has a thickness of 2.59-2.81 mm.

39. The composition of claim 29, wherein the tablet has a thickness of 2.73-3.03 mm.

40. The composition of claim 29, wherein the tablet has a thickness of 3.35-3.61 mm.

41. The composition of claim 29, wherein the tablet has a thickness of 3.88-4.24 mm.

42. The composition of claim 29, having a hardness of 4-11.2 kp.

43. The composition of claim 29, having a hardness of 4-6 kp.

44. The composition of claim 29, having a hardness of 5 kp.

45. The composition of claim 29, having a hardness of 5.7-8.6 kp.

46. The composition of claim 29, having a hardness of 7.1 kp.

47. The composition of claim 29, having a hardness of 7.4-11.2 kp.

48. The composition of claim 29, having a hardness of 9.3 kp.

49. The composition of claim 29, having a friability of 0.06-0.11%.

50. An apparatus, comprising:
an active agent comprising aripiprazole;
a substrate with a first surface and a second surface;
a partial power source comprising
a first material provided on the first surface of the substrate, wherein the first material is selected from the group consisting of magnesium, zinc, sodium, lithium, iron, and alloys thereof, an intercalation compound, vanadium oxide, manganese oxide, and combinations thereof, and
a second material provided on the second surface of the substrate, wherein the second material is selected from copper iodide, copper chloride, copper bromide, copper sulfate, copper formate, iron(III) phosphate, iron(III) pyrophosphate, oxygen, hydrogen, vanadium oxide, manganese oxide, and combinations thereof,
wherein the partial power source is configured to generate power upon contact of the first material and the second material with a fluid; and
a control unit electronically coupled with the partial power source, wherein the control unit is configured to be activated by receiving the power from the partial power source and to encode information in a current flow through the fluid.

51. A composition of matter for the ingestible administration of aripiprazole, the composition comprising:
aripiprazole; and
silicon having a mass equivalent to a silicon substrate having dimensions of between 0.2×0.2×0.2 mm (0.008 mm³) and 0.3×0.3×0.3 mm (0.027 mm³).

52. The composition of matter of claim 51, further comprising:
a metal selected from the group consisting of magnesium, zinc, sodium, lithium, iron, or alloys thereof, or combinations thereof; and
a copper salt selected from the group consisting of copper iodide, copper chloride, copper bromide, copper sulfate, copper formate, or combinations thereof.
